(19) United States
(12) Patent Application Publication
(10) Pub. No.: US 2023/0346983 A1
(43) Pub. Date: Nov. 2, 2023

(54) APPARATUS AND METHOD FOR DETECTING AND TREATING CANCEROUS TISSUE USING RAMAN SPECTROSCOPY AND HYPERTHERMIA

(71) Applicant: CytoVeris Inc., Farmington, CT (US)

(72) Inventors: Alan Kersey, South Glastonbury, CT (US); Rishikesh Pandey, Unionville, CT (US); Patrick Curry, Glastonbury, CT (US)

(21) Appl. No.: 17/796,536

(22) PCT Filed: Feb. 1, 2021
(86) PCT No.: PCT/US21/16090

§ 371 Date: Jul. 29, 2022

Related U.S. Application Data
(60) Provisional application No. 62/968,631, filed on Jan. 31, 2020.

(51) Int. Cl. A61K 49/00; A61B 5/00; A61K 41/00

(52) U.S. Cl. CPC A61K 49/001; A61B 5/0075; A61B 5/0091; A61K 41/0052

(57) ABSTRACT

A method and system for determining the presence of a mass of cancerous cells in vivo within a tissue body is provided. The method includes: a) performing an examination of the tissue body using a diagnostic method operable to determine the presence of a suspect tissue mass, and determining a location of the same; b) administering a solution containing “RR-CTEs”, the RR-CTEs configured to target and bind with cancerous cells; c) interrogating the tissue body with a beam of light, wherein the RR-CTEs are configured to produce Raman scattered light upon impingement; d) collecting the Raman scattered light; e) processing the collected Raman scattered light to determine a presence or an absence of the a Raman signature; and f) comparing the determined location of the suspect tissue mass with the determined location of the mass of cancerous cells to determine the presence of the mass of cancerous cells.

This application claims priority to U.S. Patent Appln. No. 62/968,631 filed Jan. 31, 2020, which application is hereby incorporated by reference in its entirety.

### BACKGROUND OF THE INVENTION

### 1. Technical Field

The present disclosure relates to systems and methods for the detection, identification, and/or treatment of cancerous/precancerous tissue, and more specifically systems and methods for the detection, identification, and/or treatment of cancerous tissue using Raman spectroscopy and thermal or hyperthermia treatment.

### 2. Background Information

Breast cancer diagnosis often involves using mammography to sense the presence of a tissue anomaly within a breast. Mammography uses low-dose x-rays to sense differences in tissue density. A mammogram typically cannot definitively determine whether tissue is cancerous, benign or healthy. A mammogram may also not provide clear definition of the boundaries and/or location of a suspect tissue mass. Still further, the density of healthy breast tissue can vary naturally with aging and other factors, leading to a high incidence rate of false positives reading with mammography. In the event a mammogram does indicate the presence of a suspect tissue mass, it is very often the case that a tissue biopsy will be performed to collect a tissue specimen for a histopathologic examination of the collected specimens using conventional tissue staining and microscopy. Often, to ensure that sufficient tissue is collected from the biopsy procedure, multiple biopsy needle “cores” with be taken, causing increased patient discomfort and stress. The histopathologic examination may involve a process that includes dissection, fixation, and cutting of tissue into precisely thin slices which are stained for contrast and mounted onto glass slides. The slides are examined by a pathologist under a microscope, and their interpretation of the tissue results in the pathology “read” of the sample. That process can take an extended period of time during which the patient is left to wonder. The entire process can be very traumatic to the patient.

In some instances as an alternative to mammography, breast cancer diagnosis may involve an ultrasonic examination, or a computed tomography scan (“CT scan”), or the like to sense the presence of a tissue anomaly within a breast. An ultrasonic examination utilizes high frequency sound waves to sense differences in tissue density. Many ultrasonic examination techniques do not have the ability to determine whether tissue is cancerous, benign or healthy, and there is currently limited information available regarding the value of ultrasonic examination as an early detection tool. See “Ultrasound for Breast Cancer Detection Globally A Systematic Review and Meta-Analysis”, Sood, R. et al., J Global Oncol., Aug. 27, 2019. Like a mammogram, if an ultrasonic examination does indicate the presence of a suspect tissue mass, it is very often the case that a tissue biopsy will be performed having the attendant disadvantages described above.

What is needed is a system and methodology for cancer detection that can be used in combination with other diagnostic tools such as mammography, ultrasound or the like, or independent from the same to provide meaningful cancer detection, and also a system and methodology for cancer detection that permits subsequent noninvasive treatment as required.

### SUMMARY

According to an aspect of the present disclosure, a method of determining the presence or absence of a mass of cancerous cells in vivo within a tissue body of a subject is provided. The method includes: a) performing an examination of the tissue body using a non-invasive diagnostic method operable to determine a presence or an absence of a suspect tissue mass within the tissue body, and determining a location of the suspect tissue mass determined to be present within the tissue body; b) administering a solution containing cancer targeting elements (CTEs) conjugated with Raman reporters (RR) bound to plasmonic nanoparticles, said conjugates referred to as “RR-CTEs”, wherein said RR-CTEs are configured to target and bind with cancerous cells within a predetermined period of time; c) interrogating the tissue body with a coherent beam of light impinging on an exposed skin surface of the tissue body at an impingement position after said predetermined period of time, the coherent beam of light configured to interrogate subcutaneous layers of the tissue body, wherein the RR-CTEs are configured to produce Raman scattered light with a known Raman signature upon impingement by the coherent beam of light; d) collecting the Raman scattered light at a surface of the tissue body; e) processing the collected Raman scattered light to determine a presence or an absence of the known Raman signature, wherein the presence of said Raman scattered light with the known Raman signature produced from the tissue body as a result of said impingement is indicative of the presence of said mass of cancerous cells within the interrogated tissue body, the processing including determining a location of said mass of cancerous cells within the interrogated tissue body determined to be present, and wherein the absence of said Raman scattered light with the known Raman signature produced from the tissue body as a result of said impingement is indicative of the absence of said mass of cancerous cells within the tissue body; and f) comparing the determined location of the suspect tissue mass with the determined location of the mass of cancerous cells to determine the presence of the mass of cancerous cells within the tissue body.

According to another aspect of the present disclosure, a method of treating a mass of cancerous cells in vivo within a tissue body of a subject is provided. The method includes: a) administering a solution containing cancer targeting elements conjugated with Raman reporters bound to plasmonic nanoparticles, said conjugates referred to as “RR-CTEs”, wherein said RR-CTEs are configured to target and bind with cancerous cells within a pre-determined period of time; b) interrogating the tissue body with a coherent beam of light impinging on an exposed skin surface of the tissue body at an impingement position after said predetermined period of time, the coherent beam of light configured to interrogate subcutaneous layers of the tissue body, wherein the RR-CTEs are configured to produce Raman scattered light with a known Raman signature upon impingement by the coherent beam of light; c) collecting the Raman scattered light at a surface of the tissue body; d) processing the collected Raman scattered light to determine a presence or an absence of the known Raman signature, wherein the presence of said Raman scattered light with the known Raman signature produced from the tissue body as a result of said impingement is indicative of the presence of said mass of cancerous cells within the interrogated tissue body, the processing including determining a location of said mass of cancerous cells within the interrogated tissue body determined to be present, and wherein the absence of said Raman scattered light with the known Raman signature produced from the tissue body as a result of said impingement is indicative of the absence of said mass of cancerous cells within the tissue body; and e) subjecting the tissue body at the determined location of the mass of cancerous cells with an energy configured to cause the RR-CTEs to produce a hyperthermic response sufficient to detrimentally affect the cancerous cells to which they are bound.

According to another aspect of the present disclosure, a method of treating a mass of cancerous cells in vivo within a tissue body of a subject is provided. The method comprises: a) administering a solution containing cancer targeting elements conjugated with Raman reporters, said conjugates referred to as “RR-CTEs”, wherein said RR-CTEs are configured to target and bind with cancerous cells within a pre-determined period of time; b) interrogating the tissue body with a coherent beam of light impinging on an exposed skin surface of the tissue body at an impingement position after said predetermined period of time, the coherent beam of light configured to interrogate subcutaneous layers of the tissue body, wherein the RR-CTEs are configured to produce Raman scattered light with a known Raman signature upon impingement by the coherent beam of light; c) collecting the Raman scattered light at a surface of the tissue body; d) processing the collected Raman scattered light to determine a presence or an absence of the known Raman signature, wherein the presence of said Raman scattered light with the known Raman signature produced from the tissue body as a result of said impingement is indicative of the presence of said mass of cancerous cells within the interrogated tissue body, the processing including determining a location of said mass of cancerous cells within the interrogated tissue body determined to be present, and wherein the absence of said Raman scattered light with the known Raman signature produced from the tissue body as a result of said impingement is indicative of the absence of said mass of cancerous cells within the tissue body; and e) subjecting the tissue body at the determined location of the mass of cancerous cells with an energy configured to cause the RR-CTEs to produce a hyperthermic response sufficient to detrimentally affect the cancerous cells to which they are bound.

In any of the aspects or embodiments described above and herein, the cancer targeting elements (CTEs) may be pHLIPs, and the conjugates are referred to as “RR-pHLIPs”.

In any of the aspects or embodiments described above and herein, the step of interrogating the tissue body with the coherent beam may include interrogating the tissue body with the coherent beam of light at one or more impingement positions at one or more angles relative to the skin surface.

In any of the aspects or embodiments described above and herein, the step of collecting the Raman scattered light may include collecting the Raman scattered light at one or more detector positions, each detector position separated from the impingement positions.

In any of the aspects or embodiments described above and herein, the step of processing the collected Raman scattered light to determine said presence or said absence of the known Raman signature may include creating a multidimensional map identifying spatial locations of the RR-pHLIPs disposed within the tissue body using the Raman scattered light collected at said plurality of different detector positions.

In any of the aspects or embodiments described above and herein, the Raman signature produced by the RR-pHLIPs may include at least one spectral peak in a Raman silent region.

In any of the aspects or embodiments described above and herein, the step of processing the collected Raman scattered light to determine said presence or said absence of the known Raman signature may include using a spectrometer.

In any of the aspects or embodiments described above and herein, the step of processing the collected Raman scattered light to determine said presence or said absence of the known Raman signature may include using a monochromator.

In any of the aspects or embodiments described above and herein, the step of processing the collected Raman scattered light to determine said presence or said absence of the known Raman signature may be performed without a spectrometer or a monochromator, and is performed with a light filter configured to selectively pass the known Raman signature.

In any of the aspects or embodiments described above and herein, the step of processing the collected Raman scattered light to determine said presence or said absence of the known Raman signature may include creating a multidimensional map identifying spatial locations of the RR-pHLIPs disposed within the tissue body.

In any of the aspects or embodiments described above and herein, the pHLIPs may be configured to produce the Raman scattered light with the known Raman signature upon impingement by the coherent beam of light, and the pHLIPs may be configured with at least one of an alkyne moiety, a nitrile moiety, or an azide moiety, to provide said Raman signature with at least one spectral peak in a Raman silent region.

In any of the aspects or embodiments described above and herein, each RR-pHLIP may be configured with a surface enhanced Raman spectroscopic (SERS) substrate material.

In any of the aspects or embodiments described above and herein, the method may further comprise applying energy from an electromagnetic source emitting electromagnetic radiation to the tissue body to treat the mass of cancerous cells, wherein the RR-pHLIPs are configured to absorb the electromagnetic radiation and increase in temperature to effect a hyperthermic effect in the mass of cancerous cells.

In any of the aspects or embodiments described above and herein, the electromagnetic source may emit X-ray or RF type electromagnetic radiation.

In any of the aspects or embodiments described above and herein, the step of applying energy from an electromagnetic source emitting electromagnetic radiation to the tissue body to treat the mass of cancerous cells may include monitoring Raman spectra emitted from the RR-pHLIPs and using the Raman spectra emitted from the RR-pHLIPs to determine and control a temperature of the nanoparticles.

In any of the aspects or embodiments described above and herein, the method may further comprise applying energy from a photonic source emitting photonic energy to the tissue body to treat the mass of cancerous cells, wherein the RR-pHLIPs are configured to absorb the photonic energy and increase in temperature to effect a hyperthermic effect in the mass of cancerous cells.

In any of the aspects or embodiments described above and herein, the non-invasive diagnostic method operable to determine a presence or an absence of a suspect tissue mass may utilize ultrasonic energy or mammography.

According to another aspect of the present disclosure, a system for treating a mass of cancerous cells in vivo within a tissue body of a subject is provided. The system is configured for use with a solution containing cancer targeting elements conjugated with Raman reporters bound to plasmonic nanoparticles, said conjugates referred to as “RR-CTEs”, wherein the RR-CTEs are configured to target and bind with cancerous cells within a pre-determined period of time. The system includes at least one light source, at least one light detector, and an analyzer. The at least one light source is configured to selectively emit coherent light. The at least one light detector is configured to receive light emitted from the tissue body. The analyzer is in communication with the at least one light source, the at least one detector, and a memory device storing instructions. The instructions when executed cause the analyzer to: a) control the at least one light source to interrogate subcutaneous layers of the tissue body with a coherent beam of light in a manner that the coherent beam of light impinges on an exposed skin surface of the tissue body at an impingement position, wherein the RR-CTEs are configured to produce Raman scattered light with a known Raman signature upon impingement by the coherent beam of light; b) control the at least one light detector to collect light emitted at a surface of the tissue body; c) process the collected light to determine a presence or an absence of the Raman scattered light with a known Raman signature within the collected light, wherein the presence of said Raman scattered light with the known Raman signature produced from the tissue body as a result of said impingement is indicative of the presence of said mass of cancerous cells within the interrogated tissue body, the processing including determining a location of said mass of cancerous cells within the interrogated tissue body determined to be present, and wherein the absence of said Raman scattered light with the known Raman signature produced from the tissue body as a result of said impingement is indicative of the absence of said mass of cancerous cells within the tissue body; and d) selectively subject the tissue body with an energy configured to cause the RR-CTEs to produce a hyperthermic response sufficient to detrimentally affect the cancerous cells to which they are bound at said determined location of the mass of cancerous cells found to be present within the tissue body.

In any of the aspects or embodiments described above and herein, the Raman signature produced by the RR-pHLIPs includes at least one spectral peak in a Raman silent region, and the instructions when executed cause the analyzer to process the collected light to determine a presence or an absence of the Raman scattered light with the known Raman signature within the Raman silent region.

In any of the aspects or embodiments described above and herein, the instructions when executed that cause the analyzer to process the collected emitted light to determine said presence or said absence of the Raman scattered light with said known Raman signature within the collected emitted light, further cause the analyzer to create a multidimensional map identifying spatial locations of the RR-pHLIPs disposed within the tissue body using the Raman scattered light collected at said plurality of different detector positions.

In any of the aspects or embodiments described above and herein, the instructions when executed cause the analyzer to monitor Raman spectra emitted from the RR-pHLIPs and using the Raman spectra emitted from the RR-pHLIPs to determine and control a temperature of the nanoparticles.

In any of the aspects or embodiments described above and herein, the energy configured to cause the RR-CTEs to produce a hyperthermic response sufficient to detrimentally affect the cancerous cells to which they are bound at said determined location of the mass of cancerous cells found to be present within the tissue body is produced by a source of photonic energy, and wherein the RR-pHLIPs are configured to absorb the photonic energy and react hyperthermically.

In any of the aspects or embodiments described above and herein, the instructions when executed cause the analyzer to control the at least one light source to interrogate the tissue body with the coherent beam of light at one or more points at one or more angles relative to the skin surface.

In any of the aspects or embodiments described above and herein, the system further comprising collection light optics configured to collect the emitted light at a one or more detector positions, each detector position separated from the impingement position.

In any of the aspects or embodiments described above and herein, the system further includes collection light optics, and the collection light optics include a spectrometer or a monochomator.

In any of the aspects or embodiments described above and herein, the system further includes collection light optics, and the collection light optics include a light filter configured to pass the Raman scattered light with said known Raman signature and to block other collected light without use of a spectrometer or a monochromator.

The present disclosure and advantages associated therewith will become more readily apparent in view of the detailed description provided below, including the accompanying drawings.

### BRIEF DESCRIPTION OF THE DRAWINGS

FIG. 1 is a diagrammatic illustration of a Raman reporter (RR) including a pH low insertion peptide (pHLIP) interacting with a cell membrane at neutral pH.

FIG. 2 is a diagrammatic illustration of a RR attached to a pHLIP interacting with a cell membrane in a slightly acidic environment (pH<7.0), with the pHLIP formed as a transmembrane alpha-helix inserting into the cell membrane.

FIG. 3 diagrammatically illustrates Raman spectrum associated with a Raman reporter, Raman spectrum intrinsically associated with tissue, detected Raman spectrum which is a combination thereof, and how the Raman spectrum may be processed for recognition via a spectral correlation filter.

FIG. 4A diagrammatically illustrates an RR with an enhancement moiety bonded/attached to the surface of a metallic nanoparticle at neutral pH in healthy condition.

FIG. 4B illustrates schematic of self-folding attribute of the peptide into a helix configuration in response to acidic pH in malignant conditions.

FIG. 5 represents the constituent/structural details of the RR conjugated with pHLIP.

FIG. 6 represents a graph of Raman spectral intensity versus wavenumber, illustrating a “fingerprint region” and a “silent region”

FIG. 7 represents the role of narrow band pass filter in uniquely detecting the Raman spectral signature of the enhanced moiety in the silent region. The spectrum on the left is similar to that shown in FIG. 6, and the other spectrum is primarily associated with an enhancement moiety.

FIG. 8 is a diagrammatic illustration of a system embodiment of the present disclosure.

FIG. 9 is a diagrammatic illustration of an in-vivo tissue interface device embodiment for breast examination.

FIG. 10 is a diagrammatic illustration of an in-vivo tissue interface device embodiment for breast examination.

FIG. 11 is a diagrammatic illustration of a light source angularly incident to a tissue surface, penetrating subcutaneous layers of tissue, and Raman scattered light traversing the skin surface at different lateral positions.

FIG. 12 is a flow chart illustrating an overview of aspects of the Plasmonic Raman Enhanced Deep-tissue Imaging and Cancer Theranostics Apparatus (“PREDICTA”) system and methodology.

### DETAILED DISCLOSURE

Aspects of the present disclosure are directed to a theranostic system and methodology configured to detect, define, and/or treat cancerous tissue using Raman spectroscopy techniques. As will be described herein, some embodiments of the present disclosure include hyperthermic treatment. For those embodiments of the present disclosure that include hyperthermic treatment, the present system may be referred to as “PREDICTA” (e.g., Plasmonic Raman Enhanced Deep-tissue Imaging and Cancer Theranostics Apparatus). The present disclosure includes mechanisms for specifically targeting cancer cells with highly sensitized Raman tags or “reporters” based on the plasmonic amplification of Raman signatures using nanostructures to permit detection through deep tissue layers and the subsequent treatment of the identified cancer lesion. As will be described herein, pH sensing peptides called pH (low) insertion peptides (or “pHLIPs”) are a preferred type of cancer targeting element (“CTE”). The present disclosure is not, however, limited to using pHLIPs. The present disclosure may be used as an adjunct to conventional cancer diagnostic techniques such as mammography (or ultrasound, or CT scan, or the like) or as a stand-alone system and methodology for detecting and/or treating cancerous tissue.

Cancerous tumors exhibit an acidic micro-environment, largely due to the glycolytic metabolic processes exhibited by cancer cells. More specifically, cancer cells generally exist in an acidic microenvironment having a pH of 6.4 to 6.8, whereas normal tissue typically exists in a neutral pH environment; i.e., a pH close to 7.2. To maintain the rapid growth and proliferation associated with tumor progression and tumor metastasis, cancer cells have a greater need for energy which is to a large degree fulfilled by an increased dependence on alternate metabolic pathways. Under aerobic conditions, cancer cells metabolize glucose to lactic acid, a process generally called the Warburg effect. Studies have shown that the pH in the vicinity of the plasma membrane of cancer cells is about 0.3-0.7 pH units lower than the bulk extracellular pH. Thus, cancer cells have been be described as having a “crown of acidity” near their cell surfaces (e.g., see “Applications of pHLIP Technology for Cancer Imaging and Therapy”, Trends Biotechnol. 2017 July; 35(7): 653-664). The bulk extracellular pH of tumor tissue generally correlates with perfusion, while the surface pH of cancer cells is expected to be less dependent on tumor tissue perfusion, and to be a predictive marker of tumor development and progression, since more aggressive tumor cells are more acidic.

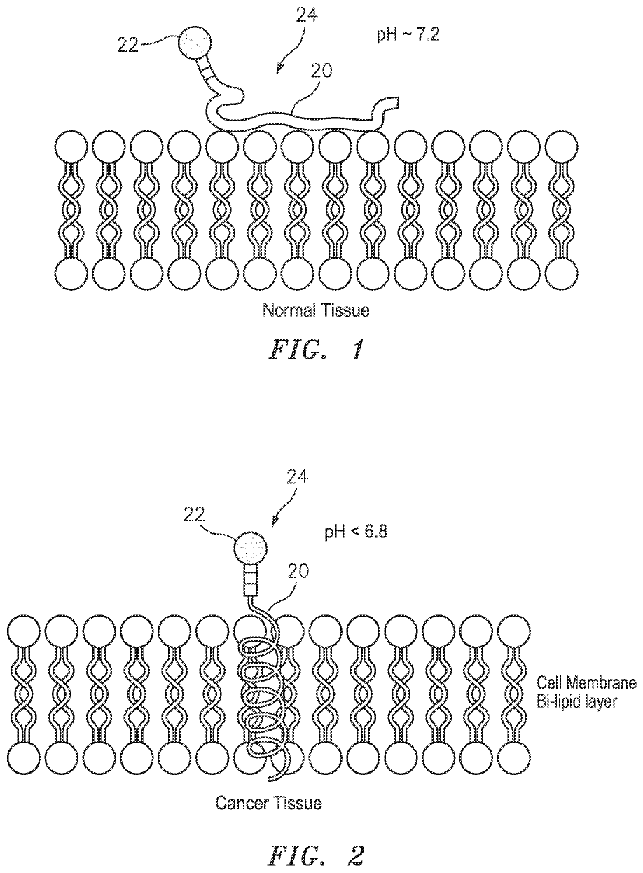

Some aspects of the present disclosure utilize pH sensing peptides called pH (low) insertion peptides 20 as a means of targeting/identifying cancer cells. pH (low) insertion peptides 20 are commonly referred to as “pHLIPs”, although it is noted that the U.S. Trademark Office issued Registration No. 3944903 for the trademark “PHLIP” for use with peptides. The term “pHLIP” as used herein refers to its common application (i.e., generically referring to pH (low) insertion peptides) and not to any particular peptide produced by any particular source. Moreover, the present disclosure may utilize a variety of different pHLIPs 20 (or other suitable cancer pH targeting methodologies as indicated herein, or as known to those in the art) and is, therefore, not limited to any particular type of pHLIP. The pHLIPs may also be engineered such that they contain unique chromophores or a functional group as an integral part of the peptide; i.e. no separate conjugation/attachment of pHLIP and a Raman reporter of (“RR”—described below) is required. pHLIPs are water-soluble membrane peptides that interact weakly with a cell membrane at neutral pH (e.g., see FIG. 1), but in a slightly acidic environment (pH<7.0) are capable of inserting into a cell membrane and forming a stable transmembrane alpha-helix (e.g., see FIG. 2). A pHLIP 20 may be used to selectively deliver a therapeutic or imaging agent to the surface of a cancer cell (e.g., the therapeutic or imaging agent is attached to a non-inserting end of the pHLIP 20), or may be used to deliver a cargo molecule to the cytoplasm of a cancer cell (e.g., the therapeutic or imaging agent is attached to an inserting end of the pHLIP 20). See “pHLIP (pH Low Insertion peptide) Technology for Cancer Diagnosis and Treatment”, Internet article at www.biophys.phys.uri.edu/pHLIP.html, Aug. 22, 2019; and “Applications of pHLIP Technology for Cancer Imaging and Therapy”, Trends Biotechnol. 2017 July; 35(7): 653-664. As indicated above, pHLIPs are a preferred element/means of targeting/identifying cancer cells (i.e., a preferred “CTE”), but the present disclosure is not limited thereto. Alternative approaches that utilize the effect of low pH tumor microenvironment to allow targeted delivery of drugs to tumor sites are known; e.g., see “Novel pH-Sensitive Cyclic Peptides”, Werakkoddy et al., Scientific Reports, 6, 2016; “Intracellular pH-sensing using core/shell silica nanoparticles”, Korzeniowska et al., J Biomed Nanotechnol., 10(7), pp. 1336-45, 2014; and 19; and “Antibody-drug conjugates: recent advances in conjugation and linker chemistries”, Tsuchikama and An, Protein Cell, 9, pp. 33-46, 2018. Photoacoustic techniques may also be used as a means of targeting/identifying cancer cells; see “A Brain Tumor Molecular Imaging Strategy Using a Triple-Modality MRI-Photoacoustic-Raman Nanoparticle”, Kircher, M. et at, Nat Med.; 18(5): 829-834. All of the articles listed above are hereby incorporated by reference in their respective entirety.

Light incident to any material has a certain probability of being scattered. As will be explained below, the present disclosure advantageously leverages Raman light scattering characteristics of materials (which distinctive scattering may be referred to as the “Raman signature” of that type of material). When photons are scattered, most of them are elastically scattered, and the scattered photons have the same energy (e.g., frequency, wavelength, color) as the incident photons but scatter in different directions. This type of photon scattering is typically referred to as “Rayleigh scattering”. Raman scattering, in contrast, refers to inelastic scattering where there is an exchange of energy and a change in the light's direction. All materials exhibit Raman scattering in response to incident light. The Raman signature of a material could be represented either as a spectrum, or as spectral images acquired at Raman scattered wavelengths. The Raman spectrum for a given material is typically complex due to the variety of molecular bonds present within the material, and the material is identifiable based on the Raman spectrum. An exemplary Raman spectrum may include a number of different peaks at a certain wavelengths or “wavenumber” offsets from incident light, which are uniquely characteristic of the material. Hence, the Raman spectrum of a particular material can be thought of as a “fingerprint” or “signature” of that particular material, and can be used for identification purposes. Human tissue has a particularly complex Raman spectrum, and the differences in the Raman spectrum associated with normal and diseased tissue can be subtle, but reproducible. The present disclosure provides a methodology for distinguishing between normal and diseased tissue despite the subtle differences in their respective Raman spectrum.

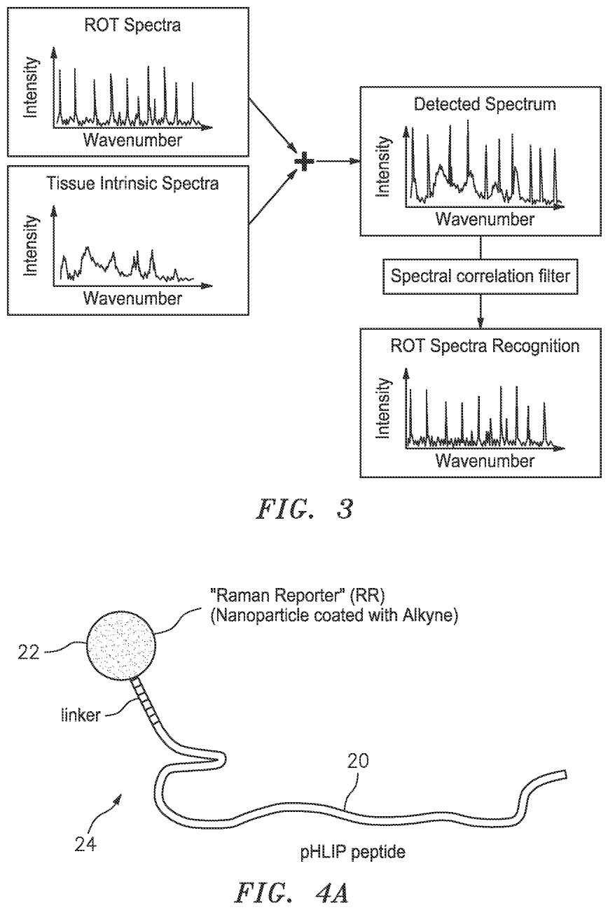

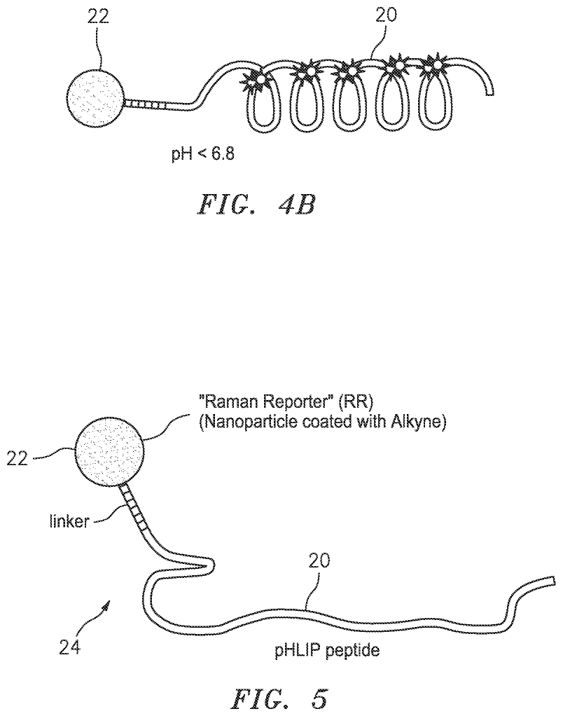

In some exemplary embodiments of the present disclosure, diseased tissue can be “tagged” with a Raman reporter (“RR”) 22 to facilitate detection of the diseased tissue. An RR 22 may be conjugated with a pHLIP 20 (collectively referred to as a “RR-pHLIP” 24), or other CTE, to create a vehicle for selectively delivering the RR 22 to the cancerous tissue (see FIGS. 4A, 4B, and 5), thereby facilitating identification of the location and geometry of the cancerous mass, and in some instances subsequent treatment of the aforesaid tissue using hyperthermic treatment. RRs 22 comprise one or more molecules often called a “Raman dye” that upon exposure to incident light at predetermined wavelengths will produce Raman scattered light with a distinct and readily identifiable signature/spectrum. FIG. 3 illustrates a Raman spectrum intrinsically associated with tissue and an exemplary “comb-like” (sometimes referred to as “code-like”) Raman spectrum from an RR, and the overall detected spectrum which is a combination thereof. FIG. 3 also illustrates how the detected spectra can be processed via a spectral correlation filter to selectively detect and analyze the spectrum associated with the RR 22. The spectral correlation filter (or similar device) may be used because the Raman spectrum of the typical RR and the tissue intrinsic spectrum overlap and reside in the same spectral region. In this manner, the RR 22 signature can be detected in the presence of otherwise interfering Raman signatures of the endogenous biospecies present. PCT publication no. WO 2020/160462 A1, commonly owned by the present applicant, provides related description, and is hereby incorporated by reference in its entirety.

In some embodiments of the present disclosure, an RR 22 may be configured with a Surface Enhanced Raman Spectroscopic (“SERS”) substrate material with one or more Raman dye molecules attached/adsorbed to the substrate surface. This substrate material is typically a metallic material, and most often takes the form of a nanoparticle or nanostructure including structures such as, but not limited to, nanostars. Upon light interrogation, SERS based RRs 22 provide Raman spectra response that is greatly enhanced relative to a Raman spectra response produced by intrinsic Raman scattering. The enhancement effects of a Raman signal is generally attributed due to the excitation of localized surface plasmons, or chemical charge transfer, or some combination thereof. The SERS effect has been demonstrated in metals such as gold and silver, as well as platinum (Pt), ruthenium (Ru), palladium (Pd), iron (Fe), cobalt (Co) and nickel (Ni). However, the SERS enhancement effect is much stronger for particles comprising a plasmonic material (e.g., noble metal, such as Au, Ag, etc.), or alkali metals (e.g., Li, Na, K, Rb, etc.), or certain base metals (e.g., Cu, etc.), or combinations or variants thereof. In some instances, an RR 22 may include novel materials such as graphene or other 2D materials that may form the basis of a SERS substrate. The nanostructure used for the SERS enhancement can either be a single metallic or a bimetallic and can be configured in various ways such as comprising a silica cell. The present disclosure is not limited to using any particular SERS material. As indicated herein, an RR 22 configured with certain types of SERS substrate material are also configured to produce a hyperthermic response as part of a hyperthermic treatment.

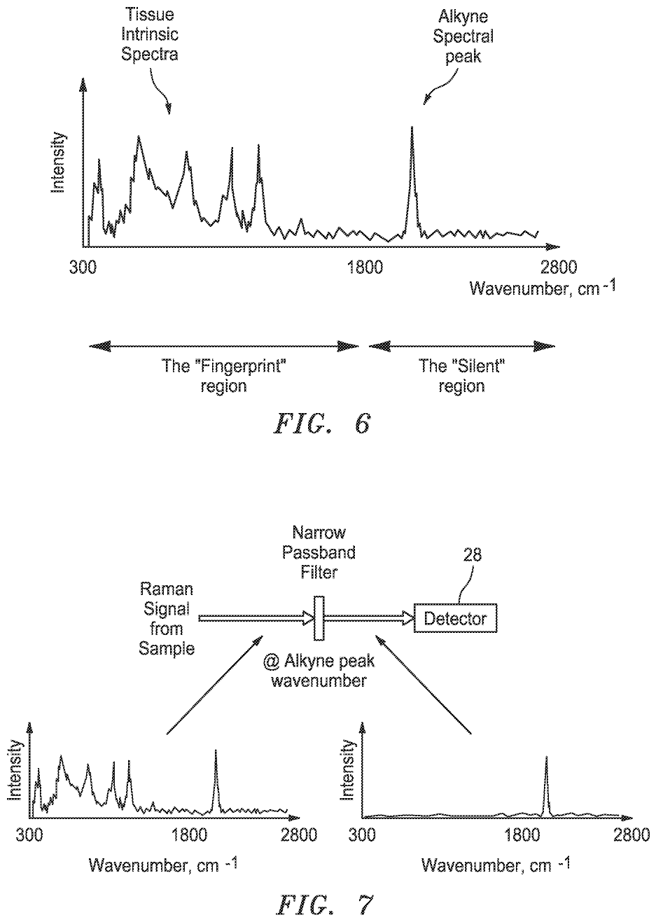

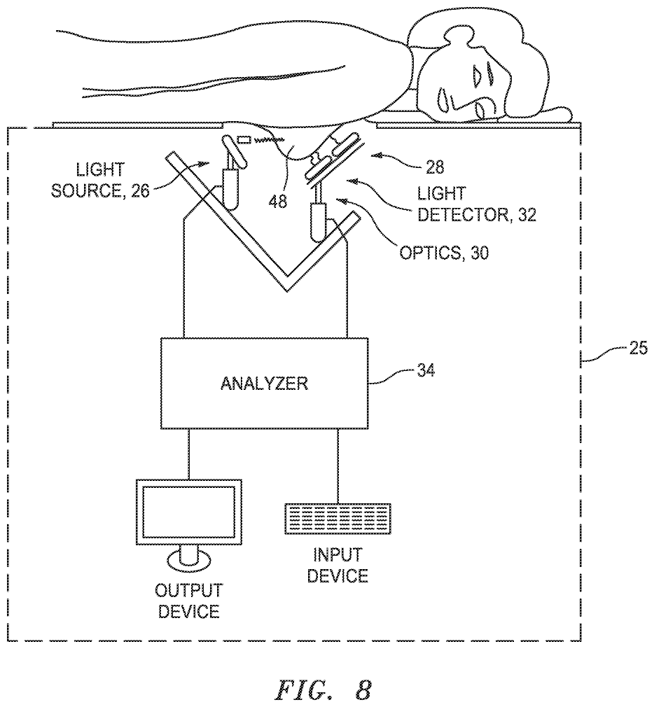

In some embodiments of the present disclosure, the RRs are configured to produce a Raman spectrum in the “Raman silent region”; i.e., a portion of the Raman spectra where the Raman spectra of endogenous biospecies (e.g., tissue intrinsic spectra, including spectra associated with tissue microcalcifications) are typically negligible. For example, an RR 22 may include an alkyne, a nitrile, or an azide moiety, or any combination thereof, or the like, bonded/attached to the surface of a nanoparticle to produce a Raman spectrum in the Raman silent region. Alkyne (a carbon-carbon triple bond) exhibits strong and characteristic peaks in the Raman silent region (typically about 1800 cm⁻¹ to 2800 cm⁻¹). In some alternative embodiments, in addition to carbon-carbon or carbon-nitrogen triple bonds, the C—H frequency of alkyne/nitrile may be used to report the presence and concentration of an alkyne/nitrile moiety. In some embodiments, polyethylene glycol (PEG) containing an alkyne moiety could be coated or otherwise attached to a nanoparticle surface. The alkyne moiety can either be a known molecular entity or a conjugated system either with a fluorophore, DNA, antibody, or any other molecular species which can act as a secondary/surrogate marker. Such surrogate markers can encode other characteristics of the targeted tissue. Non-limiting examples of these characteristics include temperature, cancer biomarker concentration, and receptor status. The depiction within FIG. 6 of tissue intrinsic spectra within both the fingerprint and silent regions, and alkyne spectral peak within the silent region illustrates well the significance of utilizing a RR 22 that produces a Raman spectrum within the silent region. In the silent region the tissue intrinsic signal intensity is negligible relative to the alkyne spectral peak, and consequently the ability to identify the RR spectrum is significantly enhanced. Furthermore, it has been discovered that utilizing a RR 22 that produces a Raman spectrum within the silent region can also simplify the means of detection. For example, some embodiments of the present disclosure may utilize a narrow-pass band filter(s) that processes only Raman spectral peak signal(s) associated with the Raman silent region (e.g., See FIG. 7). This facile filter-based detection approach facilitates an elegant Raman “imaging” system that may use a light detector 32 without the need for a spectrometer, a monochomator, or other spectral analysis device.

As will be discussed below, some RR 22 embodiments may be configured for use in a hyperthermic treatment regimen. An example of such an RR 22 is one that includes a metallic nanoparticle will absorb radiation (e.g., generated by an electromagnetic source such as a radio frequency (RF) source, or an X-ray source, or the like) at a significantly higher rate than is absorbed by tissue and will therefore increase in temperature. A specific non-limiting example of such a metallic nanoparticle is a gold nanoparticle (“AuNP”), which is known to absorb electromagnetic radiation at a significantly higher rate than tissue. As indicated above, nanoparticles such as AuNPs also provide considerable utility as a SERS enhanced particle. Hence, RRs 22 with a nanoparticle that provides SERS enhancement and is capable of producing a hyperthermic response upon excitation provide a desirable dual modality. In some embodiments, a nanoparticle may comprise multiple materials to achieve this dual modality; e.g., a first material (such as Au) for its enhanced SERS identification, and one or more other materials that are configured to produce a hyperthermic response. In still other embodiments, an RR 22 may be configured with two or more nanoparticles to provide the desired dual modality; e.g., a first nanoparticle configured to provide enhanced SERS identification and a second nanoparticle, independent of the first nanoparticle, configured to produce a hyperthermic response upon excitation from an energy source.

Other RR 22 embodiments configured for use in a hyperthermic treatment regimen may include a nanoparticle configured to produce a hyperthermic effect when subjected to an ultrasonic excitation, MRI excitation, or other excitation; e.g., see “Hyperthermia Using Nanoparticles—Promises and Pitfalls”, Kaur et al., Int J Hyperthermia. 2016; 32(1): 76-88; and “ASERRS/MRI multimodal contrast agent based on naked AU nanoparticles functionalized with a Gd(iii) loaded PEG polymer for tumor imaging and localized hyperthermia”, Litti et al., Nanoscale, 2018; 10, 1272-1278; both of which articles are hereby incorporated by reference in their respective entirety. Still further, a nanoparticle as used herein may be configured to produce a hyperthermic effect when subjected to photonic excitation; e.g., photonic excitation in the near infra-red (NIR) range (e.g., 780-1400 nm), or in the short-wave infra-red (SWIR) range (1400-3000 nm).

In those embodiments that utilize an RR 22 with a metallic SERS nanoparticle (e.g., AuNPs), the RR 22 will change its Raman spectra response (e.g., peak position, peak width, and/or response intensity, including the ratio of Raman Stokes to anti-Stokes signal) as a function of its temperature. In these embodiments, the change in Raman spectra response may be used to determine the temperature of the nanoparticle. The ability to determine the temperature of the nanoparticle can be used to control the hyperthermic treatment. In many instances, the acceptable temperature range for hyperthermic treatment is limited. A temperature below the acceptable range may not result in successful hyperthermic treatment, and a temperature above the acceptable range may result in undesirable cellular damage. Hence, the ability to control the hyperthermic treatment based on a determination of the nanoparticle temperature provides a distinct advantage.

As described above, embodiments of the present disclosure utilize pHLIPs 20 to “tag” cancerous tissue with a substance having a Raman signature that is identifiable, and that can be distinguished from local tissue Raman signals. In some embodiments of the present disclosure, a pHLIP 20 may be configured to “tag” cancerous tissue with a Raman signature that is identifiable/distinguishable from local endogenous Raman spectra without a linked RR 22. For example, in some embodiments a pHLIP 20 may be engineered to contain one or more unique chromophores or one or more functional groups as an integral part of the peptide, which chromophore or functional group produces a Raman signature that is identifiable/distinguishable from Raman spectra from cancerous and/or non-cancerous tissue. Hence, the pHLIP 20 itself may be configured to both attach to the cancerous tissue and “tag” it with a Raman signature that is identifiable/distinguishable from local Raman spectra. For example, if a pHLIP 20 is synthesized using triple carbon bonds in amino acid complexes, the “alkyne” Raman signature will be seen directly from the pHLIP 20 becoming associated with the cancerous cells. In these alternative embodiments, no separate conjugation/attachment of pHLIP 20 and a Raman reporter is required.

As another example, a pHLIP 20 may be engineered to produce a first Raman signature in its unfolded state (e.g., See FIG. 1) and to produce a second Raman signature in its folded state (e.g., See FIG. 2), which second Raman signature is distinguishable from the first Raman signature. The use of Raman spectroscopy to monitor structural changes in proteins and peptides has been reported (e.g., See Brown et al., “Bilayer surface association of the pHLIP peptide promotes extensive backbone desolvation and helically-constrained structures”, Biophysical Chemistry; 187-188, pp. 1-6, 2014; and “pHLIP (pH Low Insertion peptide) Technology for Cancer Diagnosis and Treatment”, Internet article at www.biophys.phys.uri.edu/pHLIP.html, Aug. 22, 2019, both of which articles are hereby incorporated by reference in their respective entirety).

In some embodiments of the present disclosure, alternative cancer cell targeting elements (CTEs) may be utilized. Non-limiting examples of such targeting elements include antibodies (Ab) that target certain protein biomarkers on cell membranes; e.g., EGFR, ER, CD44 HER2/Neu biomarkers. These additional cancer cell targeting elements may be utilized in combination with additional specific RR-antibody combinations and the pHLIPs 20 used to “tag” cancerous tissue with a substance having a Raman signature that is identifiable/distinguishable from Raman spectra from both the cancerous and non-cancerous tissue. The combination of the antibody targeting elements and the pHLIPs 20 with an identifiable/distinguishable Raman signal may be used to provide additional verification of the presence or absence of cancerous tissue, and/or to provide identification of one or more specific types of cancers.

Referring to FIG. 8, a diagrammatic illustration of an exemplary present disclosure system 25 embodiment is shown. The aforesaid system 25 embodiment includes a plurality of components. The system 25 embodiments include at least one light source 26, a tissue interface device (“TI device”) 28, collection light optics 30, at least one light detector 32, and an analyzer 34. As will be described herein, the configuration of these components may vary in different system 25 embodiments. The system 25 embodiment description provided herein may refer to various different system 25 components as independent components. The present disclosure is not limited to specific descriptions provided herein. For example in alternative embodiments, system 25 components may be combined, or arranged in a different manner than that shown in the Figures, and still be within the scope of the present disclosure.

The light source 26 is configured to emit coherent light. An example of an acceptable coherent light source 26 is a laser. A variety of different lasers may be used within the system 25, and the present disclosure is not therefore limited to using any particular laser. Examples of laser types include solid state, gas, diode laser or vertical-cavity surface-emitting lasers (VCSELs). The present disclosure may utilize coherent light at a variety of different wavelengths, and the light source 26 is therefore not limited to coherent light at any particular wavelength or wavelength band. The choice of a laser wavelength that permits deeper penetration into human tissue would be advantageous to the operation of the system.

The light source 26 is not limited to any particular incident beam configuration/illumination such as point, line or light-sheet. The incident beam produced by the light source 26 is configured to adequately penetrate the tissue at depths where cancerous tissue may be present within the tissue body (e.g., a breast). The ability of the present disclosure to access and treat tissue at relatively deep subcutaneous depths facilitates its ability to act as a Raman-based theranostic system and methodology. Non-limiting examples of incident beam configurations include a regular Gaussian beam, a non-diffraction Bessel beam, an Airy beam, or the like. A light source such as a Bessel beam that produces an incident beam with “self-healing” propagation properties is particularly useful because the light beam is typically able to penetrate deeper tissue depths and thereby enable analysis and/or treatment of the deeper depth tissues. As will be explained below, the orientation of the incident light relative to the tissue surface may be defined by the use of a TI device 28 such as a fixture or a probe. Hence, the light source 26 is operative to emit light, which light may pass through optical fibers and optics (e.g., lenses, mirror, filters, etc.), and then the emitted light may be oriented relative to the tissue surface by a TI device 28. The TI device 28, may allow the scanning of the incident light beam into the tissue at a variety of angles, which can be used to effectively sweep or “scan” the beam over the underlying tumor target of interest.

The collection light optics 30 are configured to transfer, and in some instances process, light emitted from the interrogated tissue; e.g., Raman scattered light emitted from the tissue as a result of incident light interrogation. The collection light optics 30 may include one or more lenses, filters, dichroic mirrors, and the like for processing the received light into a desirable form. The filters are not limited to optical filters and a filter can be any molecular system or device such as electronically-tunable acousto-optical filter as a wavelength selector. As indicated above, the filters may include one or more narrow-pass band filter(s) configured to process only wavelengths associated with defined Raman spectra peaks found within the Raman silent region (e.g., about 1800 cm⁻¹ to 2800 cm⁻¹). In addition to the band pass filters in the silent region, the present theranostic system can also be used to measure Raman intensity in the higher wavenumber region to measure single bond stretching vibrations such as C—H, O—H, and N—H vibrations. In some instances, emitted light received at the skin surface may be transferred to collection light optic 30 elements located remote from the point of collection at the skin; e.g., collected at the skin surface by optical fibers or fiber optic bundles, and transferred to remotely located collection light optic 30 elements and light detector(s) 32 (e.g., see FIG. 8). In some embodiments, as described herein, at least a portion of the collection light optics 30 may be disposed at the point of light collection on the skin (e.g., within a TI device 28 as shown in FIG. 10). The present disclosure is not limited to collection light optics 30 disposed remotely from the light collection point, or at the light collection point, and therefore contemplates any combination thereof.

The at least one light detector 32 is configured to receive light (e.g., Raman spectra) emitted from the interrogated tissue via the collection light optics 30 and produce signals representative thereof. The signals produced by the light detector 32 are transferred to the analyzer 34. Non-limiting examples of light detectors 32 include light sensors that convert light energy into an electrical signal such as a simple photodiode, or other optical detector of the type known in the art. In some embodiments, particularly those involving the use of fibers or fiber bundles to convey the light from the TI device 28 to the detectors, a charge couple device (CCD) or CMOS cameras may be used. In this case, the fibers may be arranged to fall onto an individual pixel, or groups of CCD pixels which would be “binned” into a single output. In this case a large format bandpass filter could be used to select the RR spectral feature/peak onto the CCD pixels.

The analyzer 34 is in communication with other components within the system 25, such as the at least one light source 26, the at least one light detector 32, and the like to control and or receive signals therefrom to perform the functions described herein. The analyzer 34 may include any type of computing device, computational circuit, processor(s), CPU, computer, or the like capable of executing a series of instructions that are stored in memory. The instructions may include an operating system, and/or executable software modules such as program files, system data, buffers, drivers, utilities, and the like. The executable instructions may apply to any functionality described herein to enable the system 25 to accomplish the same algorithmically and/or coordination of system 25 components. The analyzer 34 may include a single memory device or a plurality of memory devices. The present disclosure is not limited to any particular type of memory device, and may include read-only memory, random access memory, volatile memory, non-volatile memory, static memory, dynamic memory, flash memory, cache memory, and/or any device that stores digital information. The analyzer 34 may include, or may be in communication with, an input device that enables a user to enter data and/or instructions, and may include, or be in communication with, an output device configured, for example to display information (e.g., a visual display or a printer), or to transfer data, etc. Communications between the analyzer 34 and other system 25 components (e.g., the light source 26, light detector 32, etc.) may be via a hardwire connection or via a wireless connection.

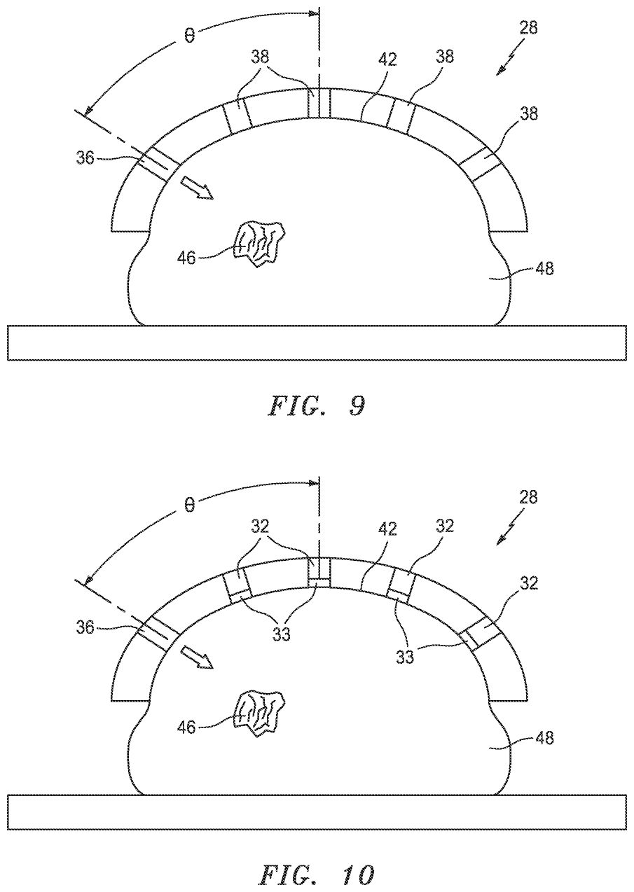

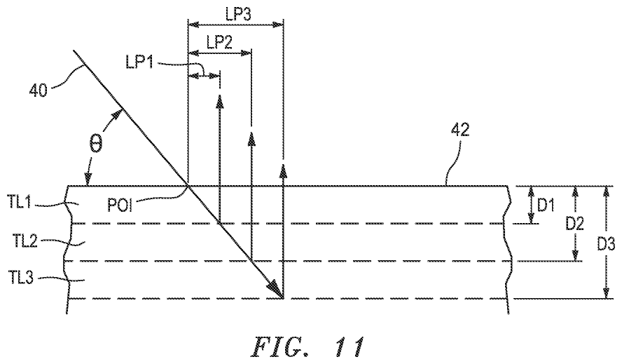

The TI device 28 is configured to position and orient light source elements (and therefore incident light) relative to the skin surface and to position and orient light collection elements relative to the skin surface. Referring to FIG. 9, in some embodiments the TI device 28 may include one or more optical fibers or other type of light conduit (referred to hereinafter as a “source fibers” 36) located at particular positions within the TI device 28, and may include a plurality of optical fibers or other type of light conduit (referred to hereinafter as a “detection fibers” 38) located at a plurality of different positions spaced apart from the incident light position(s). The source fibers 36 are in communication with the light source(s) 26 and provide a conduit for light to travel from the light source(s) 26 to the skin surface of the subject. The detection fibers 36 are in communication with the light detector(s) 32 and provide a conduit for light emitted from the tissue to travel from the skin surface of the subject to the light detector(s) 32 via the collection light optics 30. The present disclosure, including the TI device 28, is not limited to utilizing source fibers 36 and detection fibers 38. For example, as shown in FIG. 10 and/or one or more light detectors 32 may be disposed within the TI device 28. In a TI device embodiment having light detectors 32, an optical filter 33 may be positioned within the TI device 28 so as to be between the light detector 32 and the skin surface of the subject. Hence, light emitted from the subject's tissue as a result of light interrogation from the light source 26 may be filtered prior to being received by the light detector 32. In these embodiments, the optical filters 33 may be considered to be a part of the collection light optics 30. In some embodiments, the TI device 28 may include one or more light sources 26 and thereby obviate the need for respective source fibers 36. The present disclosure is not limited to the TI device 28 embodiments shown in FIGS. 9 and 10; e.g., a TI device 28 may use any combination of optical fibers, light detectors 32, and/or filters.

The TI device 28 can be configured such that light incident to the subject's skin light may be oriented in a number of different angles (e.g., an oblique angle, a perpendicular angle, etc.) relative to the skin surface. Angular orientation of the incident light can facilitate scattered light collection from different subcutaneous tissue depths. The present Raman spectroscopy techniques that utilize angularly oriented (i.e., obliquely oriented) incident light may be referred to as “angular depth resolved Raman spectroscopy” or “ADRRS”. To diagrammatically illustrate, FIG. 11 shows an incident light beam 40 impinging a skin surface 42 at a point of incidence (“POI”) at an oblique angle theta (“Θ”) relative to the skin surface 42. The incident light beam 40 penetrates tissue layers TL1, TL2, TL3 at respective subcutaneous depths D1, D2, D3. The Raman scattered light associated with tissue at depth D1 can be collected at a first lateral position LP1, the Raman scattered light associated with tissue at depth D2 can be collected at a second lateral position LP2, the Raman scattered light associated with tissue at depth D3 can be collected at a third lateral position LP3, etc. The collected light, in turn, can be used to produce a depth resolved Raman spectroscopy analysis, including a multi-dimensional representation of the tissue body. If this processing is repeated at multiple input points and angles into the tissue from the TI device 28, and the detected Raman signals recorded, the processing can take the form of a tomographic analysis, where the tumor spatial representation may be derived via a reverse problem solving or tomographic processing methodology.

In some embodiments, the TI device 28 may be shaped to conform to, or is conformable to, a breast. For example, FIGS. 8-10 diagrammatically illustrate TI devices 28 that conform to fit a breast, with incident light applied (via a source fiber 36 or a light source 26) at one or more positions and emitted light sensed (via light detectors 32 or detector fibers 38) at a plurality of positions spaced apart from the source fibers 36. The relative positions of the incident light and the detection positions, and the angle of light incidence relative to the tissue, provides Raman spectra attributable to a position and a depth within the tissue body; i.e., depth resolved Raman spectroscopy. As will be described below, the collected light may then be used to create a multi-dimensional representation (e.g., a tomographic representation) of the tissue body. The present disclosure is not limited to any particular light source and detection spatial/orientation configurations.

The following description provides an illustration of how the present disclosure Raman and hyperthermia-based theranostic system and methodology may be utilized to detect and/or treat cancerous breast tissue using Raman spectroscopy techniques. In the current standard of care, breast examinations typically involve a physical manipulation of a patient's breast to determine the presence of an abnormal tissue mass within a breast. If a potentially abnormal tissue mass is discovered, conventional practices typically call for a mammogram (or ultrasonic examination, or the like) to provide further definition of the aforesaid tissue mass to facilitate diagnosis. The mammogram results may support a diagnosis that an abnormal tissue mass is present, but mammograms typically do not provide information regarding the nature of the tissue mass; i.e., benign or malignant. If the mammogram (or ultrasound, or the like) supports a diagnosis that an abnormal tissue mass is present, under conventional practice one or more invasive tissue biopsies are performed to collect a tissue sample from the mass. The collected tissue sample(s) is then subjected to a histopathologic examination to determine whether the tissue sample is benign or malignant. If the tissue sample is malignant, then conventional treatment may involve radiation, or chemotherapy, or removal of the tissue mass, or some combination thereof.

The present disclosure Raman-based theranostic system (i.e., “PREDICTA”) and methodology provides both an attractive adjunct to conventional practices (e.g., mammograms, ultrasonic examinations, etc.), and/or an option to avoid such conventional practices and the attendant invasive tissue biopsies. If a physical breast examination suggests the presence of an abnormal tissue mass within a breast, a clinician may elect to have a mammogram (or ultrasound, or the like) done to provide further definition of the aforesaid tissue mass. If a mammogram (or ultrasound, or the like) is performed and the results supports a diagnosis that an abnormal tissue mass is present, then the present disclosure Raman-based theranostic system and methodology may be utilized as an adjunct to provide a diagnostic interpretation of the suspected abnormal tissue mass including, but not limited to, whether the tissue mass is benign or malignant, and to provide enhanced visualization of the tissue mass through the above detailed tomographic visualization. Importantly, as the pHLIP peptide targets only the malignant mass, the PREDICTA system and methodology can be utilized to determine whether the tissue mass is benign or malignant without any tissue biopsy or any histopathologic tissue sample examination. It may be said, therefore, that the PREDICTA system and methodology can serve as a “non-invasive tissue biopsy” that avoids the undesirable aspects of a conventional tissue biopsy. Furthermore if a tissue mass is determined to be malignant, then as is described herein, the present PREDICTA system and methodology provides a noninvasive treatment methodology as an alternative to conventional practices. In short, the PREDICTA system and methodology can be used with a mammogram (or ultrasound, or the like), provides a diagnostic capability that obviates the need for a conventional invasive tissue biopsy, and provides a noninvasive treatment methodology as an alternative to conventional practices. The ability to avoid tissue biopsies is significant given their invasive nature, the associated discomfort, the time required to analyze the tissue sample, and the fact that most biopsies reveal benign tissue. The present disclosure and mammography/ultrasonic examination can be used (in no required order) to non-invasively confirm findings, and to noninvasively detect the presence of cancerous tissue—thereby providing useful and confirmed information in a very short period of time prior to any invasive tissue biopsy, and utilizing the present disclosure can avoid the need for an invasive tissue biopsy altogether. This approach can be used not only for an initial periodic examination, but also for subsequent examinations as part of a treatment regimen. Hence, the present disclosure provides clinicians with a significant noninvasive tool that can be used as an adjunct to conventional diagnostic techniques to provide quick and accurate noninvasive results. To be clear, some clinicians may still wish to utilize invasive biopsies for still further confirmation, and the present disclosure does not prevent such procedures.

Alternatively, the present disclosure system and methodology can provide an attractive stand-alone noninvasive system and methodology—it does not require a conventional diagnostic technique such as mammography, ultrasonic examination, or the like. It can be used by itself to noninvasively detect the presence of cancerous tissue in a very short period of time without the need for invasive tissue biopsies. As stated above, the present system and method can be used for an initial periodic examination, and also for subsequent examinations as part of a treatment regimen. Hence, the present disclosure provides clinicians with a significant, novel, and unobvious noninvasive tool that can be used to provide quick and accurate results and treatment as may be required. Owing to its accurate cancer targeting and detection attributes, the present disclosure system and methodologies can be utilized, at a minimum, to take fewer high-quality tissue biopsies, thereby minimizing the discomfort and the need for multiple patient visits in some cases.

Under the present disclosure the PREDICTA system and methodology, once the presence of an abnormal tissue mass within a breast is suspected (regardless of whether it has been confirmed by a mammogram), a clinician may utilize the present PREDICTA system and methodology to create a meaningful diagnostic interpretation of the suspected tissue. A material containing RR-pHLIPs 24 (or RR 22 having an alternative CTE) may be administered to the patient. The material may be configured in different forms (e.g., a fluid, a solid, etc.) and may be administered in a variety of different ways (e.g., intravenously, orally, topically, etc.). Some number of those RR-pHLIPs 24 will in time reside in an acidic environment produced on or in close proximity to the surface of a cancer cell. In that acidic environment, at least some of those RR-pHLIPs 24 will form an alpha helix configuration that links the respective RR-pHLIP 24 with a respective cancer cell. Those RR-pHLIPs 24 that do not link with a cancer cell will naturally purge from the patient's system over a determinable period of time. After a period of time sufficient for the RR-pHLIPs 24 to link with any cancer cells that may be present and for unbound RR-pHLIPs 24 to be purged, the patient's breast may be interrogated using the present system 25; e.g., using a TI device 28 in communication with the suspect breast.

During operation of the system 25, the light source 26 controlled within the system 25 produces incident light that will penetrate the breast tissue at depths sufficient to interrogate the suspected tissue mass 46 (see FIGS. 8 and 9) within the breast 48. Incident light interacting with the RRs 22 linked to cancer cells by pHLIPs 20 will produce strong Raman scattered light. As stated above, the Raman scattered light traversing to the surface of the breast may be collected by detector fibers 38 and passed through the collection light optics 30 prior to reaching the light detectors 32, or it may pass though optical filters (for optically selecting the silent region) 33 and be collected directly by light detectors 32 directly placed on the tissue surface.

The collected Raman spectra provides a distinct photometric signature indicative of the RRs 22. As indicated above, RRs 22 utilized with the present disclosure may be configured with a Raman dye (e.g., including an alkyne moiety) that produces a Raman spectrum within the Raman silent region. In the silent region, the Raman signal produced by tissue and other abnormal conditions (e.g., calcifications) is negligible relative to the Raman spectra produced by the aforesaid RR 22. Hence, Raman spectrum produced by the RR 22 is clearly identifiable in the Raman silent region. In those embodiments wherein the RR includes a SERS substrate material (e.g., an AuNP), the Raman spectrum produced by the RR 22 will be greatly enhanced relative to the Raman spectrum produced by a non-SERS RR 22. The enhancement/amplification of the Raman spectrum produced by a SERS modified RR 22 is particularly useful when the present PREDICTA system is utilized to detect, analyze, and treat deep tissue applications such as cancerous tissue masses deep within breast tissue.

In some exemplary embodiments, the system 25 can use filtering (e.g., narrow-pass bandwidth filters) to ascertain the presence or absence of the aforesaid Raman spectra in the Raman silent region without the need for a spectrometer, a monochromator, or other similar functioning device. The optical filters described above can be configured as narrow-pass bandwidth filters configured to pass Raman spectra in the Raman silent region; e.g., Raman spectra associated with alkyne or nitriles. The ability of the present PREDICTA system and methodology to provide diagnostic information without a spectrometer, a monochromator, or similar device provides a significant improvement over the prior art, as the optical “throughput” (e.g., overall optical light collection efficiency) is greatly improved. The light detector 32 produces signals representative of the collected light and communicates those signals to the analyzer 34.

As stated above, the analyzer 34 is in communication with other system 25 components (e.g., the light source 26, the light detector 32, etc.) to control and or receive signals therefrom to perform the functions described herein. During operation of the present disclosure system 25, the analyzer 34 is configured to execute stored instructions that cause the light source 26 and the light detector 32 to operate in the manner described herein. Also during operation of the system 25, the analyzer 34 is configured to execute stored instructions for processing the signals received from the light detector 32.

In those exemplary embodiments wherein the present system is configured to interrogate the subject tissue from a plurality of distances, at different angles of incident light orientation (e.g., oblique angles), etc., the signals produced by the light detector 32 provide multi-dimensional information (e.g., positional and depth information) relating to the interrogated tissue. The analyzer 34 processes the aforesaid signals to provide a multi-dimensional mapping of the cancerous tissue using tomographic processing methodologies, which are typically based on “reverse problem solving” algorithms; e.g., a three-dimensional tomographic map or image of the cancerous tissue based on RR-pHLIPs 24 linked to the cancerous tissue. If a suspect tissue mass 46 did not in fact comprise cancerous tissue, analysis results provided by the analyzer 34 would so indicate. The use of RR-pHLIPs 24 make the analysis of the present system 25 specific to cancerous tissue. Mammogram (or ultrasound, or the like) results, in contrast, are typically not cancer specific; e.g., a mass of tissue having an abnormal density within a breast may appear within mammogram results to be potentially cancerous, but the mammogram results are typically not cancer definitive. To get definitive cancer information, it is often necessary to perform an invasive biopsy. The present disclosure obviates the need for the invasive biopsy.

In the event a cancerous tissue mass 46 is identified, the present disclosure Raman-based theranostic system and methodology is configured to permit radiation treatment of the cancerous tissue mass. The RR-pHLIPs 24 linked to the cancerous tissue may be initially utilized as a targeting mechanism for the radiation treatment. For example, the tomographic representation of the cancerous tissue mass 46 created during detection, may now be used to target an application of radiation. The cancerous tissue mass 46 can be subjected to one or more applications of radiation that cause the nanoparticle portions of the RR-pHLIPs 24 linked to the cancer cells to increase in temperature to a level where they detrimentally affect the cancer cell. More specifically, the applied radiation causes the metallic nanoparticle of each RR 22 (e.g., an Au NP) to increase in temperature to a level where it detrimentally affects the cancer cell to which it is connected via the pHLIP 20. The aforesaid process (including the cancerous tissue mass detection and subsequent radiation treatment) can be periodically performed numerous times; e.g., until the cancerous tissue mass 46 is no longer present. Indeed, using the present system and method it may be possible to avoid invasive surgical removal of cancerous tissue. As indicated herein, some embodiments of the present disclosure include techniques for determining and controlling the temperature of RRs 22, and thereby control the hyperthermic treatment process.

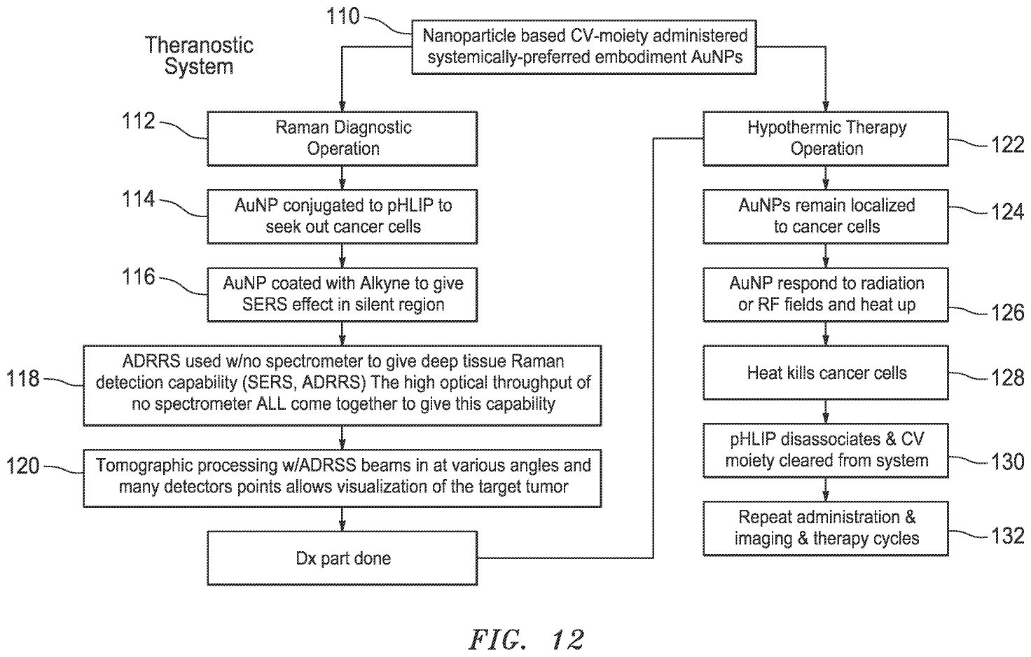

FIG. 12 provides a useful overview of aspects of the present PREDICTA theranostic system and methodology. The flow chart shown in FIG. 12 begins with a solution containing pHLIPS 20 configured to “tag” cancerous tissue with a Raman signature that is identifiable/distinguishable from local endogenous Raman spectra; e.g., “tagged” pHLIPs are administered to a subject (step 110). In a first diagnostic path 112 of the present theranostic methodology, the tagged pHLIPs pass within the subject's body linking with cancer cells where present (step 114). The suspect tissue area of the subject may be photometrically interrogated with incident laser light in a manner described above as “angular depth resolved Raman spectroscopy” (ADRRS), but is not limited thereto. Alternatively, a variety of different interrogation techniques may be used wherein the light may be angularly modulated relative to the tissue. The interaction between the incident light and the tagged pHLIPs (e.g., tagged pHLIPS bearing an Alkyne or similar moiety) will produce distinct Raman spectra within the Raman silent region. The Raman spectra within the Raman silent region can be identified and processed without the use of a spectrometer. (Steps 116, 118) The light interrogation process can be performed with a plurality of light beams at different angles and a plurality of light detectors to produce ADRRS data that enables three-dimensional visualization and/or mapping of the identified cancerous tissue mass. (Step 120).

Once the diagnostic portion of the methodology is performed (“Dx part done”), and that diagnostic portion may include a mammogram, an ultrasonic examination, or the like, the hyperthermic treatment path (step 122) may be performed. RR-pHLIPs 24 administered to a subject will remain linked to the cancerous cells for a useful period of time (step 124), which may span the time necessary for the mammogram/ultrasound examination, and the present diagnostic portion of the present disclosure to be performed as an adjunct. If a significant amount of time is expended between the mammogram/ultrasound examination, and the present diagnostic portion of the present disclosure, then it may be necessary to re-administer the material containing the RR-pHLIPs to the patient. Regardless, once the diagnostic process is completed and the material containing the RR-pHLIPs has been administered to the subject, electromagnetic radiation (e.g., RF, X-ray, etc.) or other energy (e.g., photonic) is targeted at the identified cancerous tissue mass and therefore also at the RR-pHLIPs 24 linked to cells within the cancerous tissue mass. The radiation causes the linked RRs 22 to increase to a temperature that will kill the linked cancer cell (steps 126, 128). As stated above, embodiments of the present disclosure may include techniques for controlling the temperature of the RRs 22 and therefore the hyperthermic treatment process. Over a short period of time, the pHLIP linking each respective RR-pHLIP 24 to a cell will disassociate with the aforesaid cell, and the RR-pHLIP 24 will purge from the subject's system (step 130). The aforesaid processes of administering a solution containing pHLIP-conjugated SERS reporters, ADRRS imaging the subject to produce determine three-dimensional visualization and/or mapping of the identified cancerous tissue mass, and treating the cancerous cells (e.g., using electromagnetic or photonic energy) can be repeated a plurality of times as necessary (step 132).

The present disclosure PREDICTA system and methodology represents numerous significant advancements over conventional breast cancer detection and treatment. Conventional breast cancer detection techniques typically require a biopsy to collect a tissue sample and a histopathologic examination of that sample to determine whether the tissue is benign or malignant. As stated above, these conventional invasive techniques can cause physical discomfort and emotional stress given the time required to analyze the tissue sample. Moreover, most of these biopsies reveal benign tissue. The present disclosure Raman-based theranostic system and methodology, in contrast, can provide a diagnostic interpretation of the suspected abnormal tissue mass that obviates the need for any tissue biopsy or any histopathologic tissue sample examination. The present disclosure provides significant cancer targeting and detection via RRs 22 conjugated with one or more pHLIPs 20. The utilization within the present disclosure of SERS enhanced RRs 22 with an enhancement moiety provides a significant improvement in detection relative to any known Raman analysis techniques. As stated above, the SERS enhanced RRs 22 provide a significantly enhanced Raman spectra response, and a Raman dye that produces Raman spectra in the Raman silent region (e.g., an alkyne moiety) creates a Raman spectra response that is readily distinguishable from other Raman spectra response associated with endogenous biospecies.

While the principles of the disclosure have been described above in connection with specific apparatuses and methods, it is to be clearly understood that this description is made only by way of example and not as limitation on the scope of the disclosure.

The singular forms “a,” “an,” and “the” refer to one or more than one, unless the context clearly dictates otherwise. For example, the term “comprising a specimen” includes single or plural specimens and is considered equivalent to the phrase “comprising at least one specimen.” The term “or” refers to a single element of stated alternative elements or a combination of two or more elements, unless the context clearly indicates otherwise. As used herein, “comprises” means “includes.” Thus, “comprising A or B,” means “including A or B, or A and B,” without excluding additional elements. Further, the term “coupled” does not exclude the presence of intermediate elements between the coupled items. Also, any reference to attached, fixed, connected or the like may include permanent, removable, temporary, partial, full and/or any other possible attachment option.

It is noted that various connections are set forth between elements in the present description and drawings (the contents of which are included in this disclosure by way of reference). It is noted that these connections are general and, unless specified otherwise, may be direct or indirect and that this specification is not intended to be limiting in this respect. A coupling between two or more entities may refer to a direct connection or an indirect connection. An indirect connection may incorporate one or more intervening entities or a space/gap between the entities that are being coupled to one another.

Furthermore, no element, component, or method step in the present disclosure is intended to be dedicated to the public regardless of whether the element, component, or method step is explicitly recited in the claims. No claim element herein is to be construed under the provisions of 35 U.S.C. 112(f) unless the element is expressly recited using the phrase “means for.”

While various inventive aspects, concepts and features of the disclosures may be described and illustrated herein as embodied in combination in the exemplary embodiments, these various aspects, concepts, and features may be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein. For example, in the exemplary embodiments described above within the Detailed Description portion of the present specification, elements are described as individual units and shown as independent of one another to facilitate the description. In alternative embodiments, such elements may be configured as combined elements.

Additionally, even though some features, concepts, or aspects of the disclosures may be described herein as being a preferred arrangement or method, such description is not intended to suggest that such feature is required or necessary unless expressly so stated. Still further, exemplary or representative values and ranges may be included to assist in understanding the present application, however, such values and ranges are not to be construed in a limiting sense and are intended to be critical values or ranges only if so expressly stated.

Descriptions of exemplary methods or processes are not limited to inclusion of all steps as being required in all cases, nor is the order that the steps are presented to be construed as required or necessary unless expressly so stated. The words used in the claims have their full ordinary meanings and are not limited in any way by the description of the embodiments in the specification.

What is claimed:

1. A method of determining the presence or absence of a mass of cancerous cells in vivo within a tissue body of a subject, the method comprising:
performing an examination of the tissue body using a non-invasive diagnostic method operable to determine a presence or an absence of a suspect tissue mass within the tissue body, and determining a location of the suspect tissue mass determined to be present within the tissue body;
administering a solution containing cancer targeting elements (CTEs) conjugated with Raman reporters (RR), said conjugates referred to as “RR-CTEs”;
wherein said RR-CTEs are configured to target and bind with cancerous cells within a predetermined period of time;
interrogating the tissue body with a coherent beam of light impinging on an exposed skin surface of the tissue body at an impingement position after said predetermined period of time, the coherent beam of light configured to interrogate subcutaneous layers of the tissue body;
wherein the RR-CTEs are configured to produce Raman scattered light with a known Raman signature upon impingement by the coherent beam of light;
collecting the Raman scattered light at a surface of the tissue body;
processing the collected Raman scattered light to determine a presence or an absence of the known Raman signature, wherein the presence of said Raman scattered light with the known Raman signature produced from the tissue body as a result of said impingement is indicative of the presence of said mass of cancerous cells within the interrogated tissue body, the processing including determining a location of said mass of cancerous cells within the interrogated tissue body determined to be present, and wherein the absence of said Raman scattered light with the known Raman signature produced from the tissue body as a result of said impingement is indicative of the absence of said mass of cancerous cells within the tissue body; and
comparing the determined location of the suspect tissue mass with the determined location of the mass of cancerous cells to determine the presence of the mass of cancerous cells within the tissue body.

2. The method of claim 1, wherein the cancer targeting elements are pHLIPs, and the conjugates are referred to as “RR-pHLIPs”, and the step of interrogating the tissue body with the coherent beam includes interrogating the tissue body with the coherent beam of light at one or more impingement positions at one or more angles relative to the skin surface.

3. (canceled)

4. The method of claim 2, wherein the step of collecting the Raman scattered light includes collecting the Raman scattered light at one or more detector positions, each detector position separated from the impingement positions.

5. (canceled)

6. The method of claim 2, where the Raman signature produced by the RR-pHLIPs includes at least one spectral peak in a Raman silent region.

7. The method of claim 2, wherein the step of processing the collected Raman scattered light to determine said presence or said absence of the known Raman signature includes using a spectrometer.

8. The method of claim 2, wherein the step of processing the collected Raman scattered light to determine said presence or said absence of the known Raman signature is performed without a spectrometer or a monochromator, and is performed with a light filter configured to selectively pass the known Raman signature.

9. The method of claim 2, wherein the step of processing the collected Raman scattered light to determine said presence or said absence of the known Raman signature includes creating a multidimensional map identifying spatial locations of the RR-pHLIPs disposed within the tissue body.

10. (canceled)

11. The method of claim 1, wherein the Raman reporters (RR) are bound to plasmonic nanoparticles.

12-18. (canceled)

19. A method of treating a mass of cancerous cells in vivo within a tissue body of a subject, the method comprising:
administering a solution containing cancer targeting elements conjugated with Raman reporters bound to plasmonic nanoparticles, said conjugates referred to as “RR-CTEs”;
wherein said RR-CTEs are configured to target and bind with cancerous cells within a pre-determined period of time;
interrogating the tissue body with a coherent beam of light impinging on an exposed skin surface of the tissue body at an impingement position after said predetermined period of time, the coherent beam of light configured to interrogate subcutaneous layers of the tissue body;
wherein the RR-CTEs are configured to produce Raman scattered light with a known Raman signature upon impingement by the coherent beam of light;
collecting the Raman scattered light at a surface of the tissue body;
processing the collected Raman scattered light to determine a presence or an absence of the known Raman signature, wherein the presence of said Raman scattered light with the known Raman signature produced from the tissue body as a result of said impingement is indicative of the presence of said mass of cancerous cells within the interrogated tissue body, the processing including determining a location of said mass of cancerous cells within the interrogated tissue body determined to be present, and wherein the absence of said Raman scattered light with the known Raman signature produced from the tissue body as a result of said impingement is indicative of the absence of said mass of cancerous cells within the tissue body; and
subjecting the tissue body at the determined location of the mass of cancerous cells with an energy configured to cause the RR-CTEs to produce a hyperthermic response sufficient to detrimentally affect the cancerous cells to which they are bound.

20. The method of claim 19, wherein the cancer targeting elements are pHLIPs, and the conjugates are referred to as “RR-pHLIPs”, and the step of interrogating the tissue body with the coherent beam includes interrogating the tissue body with the coherent beam of light at one or more impingement positions at one or more angles relative to the skin surface.

21-23. (canceled)

24. The method of claim 20, where the Raman signature produced by the RR-pHLIPs includes at least one spectral peak in a Raman silent region.

25. The method of claim 19, wherein the step of processing the collected Raman scattered light to determine said presence or said absence of the known Raman signature includes using a spectrometer.

26. The method of claim 19, wherein the step of processing the collected Raman scattered light to determine said presence or said absence of the known Raman signature is performed without a spectrometer or a monochromator, and is performed with a light filter configured to selectively pass the known Raman signature.

27. The method of claim 20, wherein the step of processing the collected Raman scattered light to determine said presence or said absence of the known Raman signature includes creating a multidimensional map identifying spatial locations of the RR-pHLIPs disposed within the tissue body.

28-33. (canceled)

34. The method of claim 20, wherein the step of subjecting the tissue body at the determined location of the mass of cancerous cells with said energy includes applying said energy from a photonic source emitting photonic energy to the tissue body to treat the mass of cancerous cells, wherein the RR-pHLIPs are configured to absorb the photonic energy and increase in temperature to effect a hyperthermic effect in the mass of cancerous cells.

35. (canceled)

36. A system for treating a mass of cancerous cells in vivo within a tissue body of a subject, the system for use with a solution containing cancer targeting elements conjugated with Raman reporters, said conjugates referred to as “RR-CTEs”, wherein the RR-CTEs are configured to target and bind with cancerous cells within a pre-determined period of time, the system comprising:
at least one light source configured to selectively emit coherent light;
at least one light detector configured to receive light emitted from the tissue body; and
an analyzer in communication with the at least one light source, the at least one detector, and a memory device storing instructions, which instructions when executed cause the analyzer to:
control the at least one light source to interrogate subcutaneous layers of the tissue body with a coherent beam of light in a manner that the coherent beam of light impinges on an exposed skin surface of the tissue body at an impingement position;
wherein the RR-CTEs are configured to produce Raman scattered light with a known Raman signature upon impingement by the coherent beam of light;
control the at least one light detector to collect light emitted at a surface of the tissue body; and
process the collected light to determine a presence or an absence of the Raman scattered light with a known Raman signature within the collected light, wherein the presence of said Raman scattered light with the known Raman signature produced from the tissue body as a result of said impingement is indicative of the presence of said mass of cancerous cells within the interrogated tissue body, the processing including determining a location of said mass of cancerous cells within the interrogated tissue body determined to be present, and wherein the absence of said Raman scattered light with the known Raman signature produced from the tissue body as a result of said impingement is indicative of the absence of said mass of cancerous cells within the tissue body; and
selectively subject the tissue body with an energy configured to cause the RR-CTEs to produce a hyperthermic response sufficient to detrimentally affect the cancerous cells to which they are bound at said determined location of the mass of cancerous cells found to be present within the tissue body.

37. The system of claim 36, wherein the cancer targeting elements are pHLIPs, and the conjugates are referred to as “RR-pHLIPs”, wherein the pHLIPs are configured to produce the Raman scattered light with the known Raman signature upon impingement by the coherent beam of light, and the pHLIPs are configured to provide said known Raman signature with at least one spectral peak in a Raman silent region.

38-39. (canceled)

40. The system of claim 37, wherein the energy configured to cause the RR-CTEs to produce a hyperthermic response sufficient to detrimentally affect the cancerous cells to which they are bound at said determined location of the mass of cancerous cells found to be present within the tissue body is produced by a source of electromagnetic radiation, and wherein the RR-pHLIPs are configured to absorb the electromagnetic radiation and react hyperthermically.

41-42. (canceled)

43. The system of claim 37, wherein the cancer targeting elements are pHLIPs, and the conjugates are referred to as “RR-pHLIPs”, and wherein the Raman signature produced by the RR-pHLIPs includes at least one spectral peak in a Raman silent region, and the instructions when executed cause the analyzer to process the collected light to determine a presence or an absence of the Raman scattered light with the known Raman signature within the Raman silent region.

44. The system of claim 37, wherein the instructions when executed that cause the analyzer to process the collected emitted light to determine said presence or said absence of the Raman scattered light with said known Raman signature within the collected emitted light, further cause the analyzer to create a multidimensional map identifying spatial locations of the RR-pHLIPs disposed within the tissue body using the Raman scattered light collected at said plurality of different detector positions.

45. The system of claim 37, wherein the instructions when executed cause the analyzer to monitor Raman spectra emitted from the RR-pHLIPs and using the Raman spectra emitted from the RR-pHLIPs to determine and control a temperature of the nanoparticles.

46-51. (canceled)
